(19) United States
(12) Patent Application Publication
(10) Pub. No.: US 2014/0044759 A1
(43) Pub. Date: Feb. 13, 2014

(54) DRUG SUBSTANCES, PHARMACEUTICAL COMPOSITIONS AND METHODS FOR PREPARING THE SAME

(71) Applicants: Dimitrios Zarkadas, Fanwood, NJ (US); Christopher Pridgen, Weehawken, NJ (US); Vincenzo Liotta, Glen Ridge, NJ (US)

(72) Inventors: Dimitrios Zarkadas, Fanwood, NJ (US); Christopher Pridgen, Weehawken, NJ (US); Vincenzo Liotta, Glen Ridge, NJ (US)

(21) Appl. No.: 14/114,357

(22) PCT Filed: May 2, 2012
(86) PCT No.: PCT/US12/36131

§ 371 Date: Oct. 28, 2013

Related U.S. Application Data
(60) Provisional application No. 61/482,592, filed on May 4, 2011.

(51) Int. Cl. A61K 9/00; A61K 38/06; C07K 1/36; A61K 38/00

(52) U.S. Cl. CPC A61K 9/00; A61K 38/005; A61K 38/06; C07K 1/36
USPC 424/400; 514/4.3; 530/331; 428/219

(57) ABSTRACT

Drug substances, which comprise a solid amorphous forms of a compound of structural formula I and have a BET specific surface area of up to about 94 m²/g, pharmaceutical compositions comprising such drug substances, processes for preparing such drug substances and uses of such drug substances and pharmaceutical compositions are disclosed.

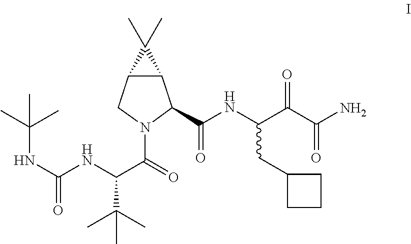 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

### FIELD OF THE INVENTION

This disclosure relates to drug substances comprising inhibitors of the hepatitis C virus (HCV) and having advantageous properties, pharmaceutical compositions comprising such drug substances, methods of preparing such drug substances, and methods of treating hepatitis C viral infection with such drug substances.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B). The NS3 protease is located in the N-terminal domain of the NS3 protein, and is considered a prime drug target because it is responsible for an intramolecular cleavage at the NS3/4A site and for downstream intermolecular processing at the NS4A/4B, NS4B/5A and NS5A/5B junctions.

U.S. Pat. No. 7,012,066 describes compounds that are useful as HCV NS3 inhibitors and useful in the treatment of HCV and conditions caused by HCV infection. One such inhibitor of the HCV non-structural protein 3 (NS3) serine protease is boceprevir. Boceprevir has the chemical name (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide. The structure of boceprevir is

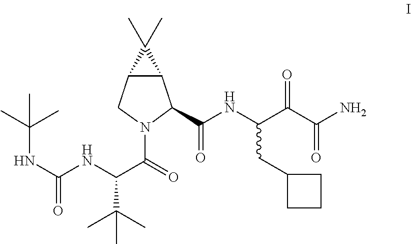 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

U.S. Pat. Nos. 7,728,165, 7,723,531, 7,595,419, 7,569,705, 7,528,263, 7,326,795, 7,309,717, and 6,992,220; U.S. Patent Application Publications No. US2011/0034705, US2010/0256393, US2010/0145069, US2010/0145013, US2010/0113821, US2009/0326244, US2008/0254128, and US2008/0193518; and International Patent Application Publication WO2009/073380 describe processes for preparing such compounds and/or for preparing drug products containing such compounds, including processes for preparing particles for pharmaceutical formulations having specified physical attributes. U.S. Pat. No. 7,772,178 describes pharmaceutical formulations comprising boceprevir. The disclosures of each of the above-mentioned patents and publications are incorporated in their entirety.

However, there is a continuing need for improved processes for preparing drug substances, compositions and formulations containing compounds that are potent inhibitors of intermolecular cleavage at the NS3/4A site. This disclosure addresses this need.

### SUMMARY OF THE INVENTION

Boceprevir is manufactured and sold as an encapsulated solid dosage form. The boceprevir drug substance is an approximately equal mixture of diastereomers of the compound of structural formula I:

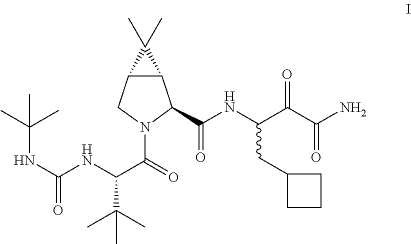 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

The compound has five chiral centers, four of which are controlled during the manufacturing process. The remaining chiral center is controlled to produce an approximately 1:1 mixture of diastereomers. The boceprevir diastereomer mixture is amorphous.

As a solid dosage form, dissolution is an important performance attribute in achieving bioavailability for oral administration. Specific surface area of the drug substance has a significant effect on the dissolution of boceprevir. It is a key physiochemical property that must be controlled to ensure satisfactory in vivo dissolution of boceprevir.

The present invention relates to drug substances having defined specific surface areas, pharmaceutical compositions comprising such drug substances, and processes for preparing such drug substances.

Other embodiments, aspects and features of the present invention are either further described in or will be apparent from the ensuing description, examples and appended claims.

### BRIEF DESCRIPTION OF THE DRAWINGS

FIG. 1 is a schematic representation of the process of drug substance isolation.

FIG. 2 is a cross-sectional schematic view of a Tee-fitting apparatus useful for preparing a slurry in accordance with the claimed invention.

FIG. 3 is a graphic representation of the correlation between drug substance BET specific surface area prior to distillation and drug substance BET specific surface area following distillation according to the claimed process of drug substance isolation, according to Example 3.

FIG. 4 is a graphic representation of the changes to BET specific surface area during processing according to the claimed process of drug substance isolation, according to Example 3.

FIG. 5 is a graphic representation of the information provided in Table 2 and in Example 3.

FIG. 6 is a graphic representation of drug substance BET specific surface area over time during processing according to the claimed process of drug substance isolation, according to Example 10.

FIGS. 7A and 7B illustrate the morphology of boceprevir drug substance prepared by the process of Example 8.

FIG. 8 is a graphic representation of drug substance BET specific surface area as a function of time for different equilibration temperatures, according to Example 9.

FIG. 9 is a graphic representation of the effect of thermal history on drug substance BET specific surface area, according to Example 11.

FIG. 10 is a graphic representation of the effect of composition of drug substance BET specific surface area, according to Example 12.

### DETAILED DESCRIPTION OF THE INVENTION

A first embodiment of the invention relates to a drug substance comprising a compound of structural formula I:

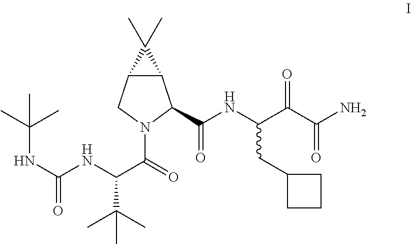 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

wherein the drug substance is solid and the drug substance has a BET specific surface area of from about 2.9 m²/g to about 94 m²/g. In aspects of this first embodiment, the drug substance has a BET specific surface area of from about 2.9 m²/g to about 9.6 m²/g. In specific aspects of this first embodiment, the drug substance has a BET specific surface area of from about 2.9 m²/g to about 9.4 m²/g.

A second embodiment of the invention relates to a pharmaceutical composition comprising at least one drug substance and at least one pharmaceutically acceptable carrier, where the at least one drug substance comprises a compound of structural formula I:

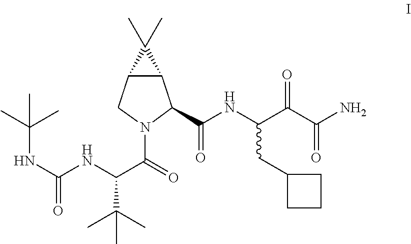 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

wherein the drug substance is solid and the drug substance has a BET specific surface area of from about 2.9 m²/g to about 94 m²/g. In aspects of this second embodiment, the drug substance has a BET specific surface area of from about 2.9 m²/g to about 9.6 m²/g. In specific aspects of this second embodiment, the drug substance has a BET specific surface area of from about 2.9 m²/g to about 9.4 m²/g. In additional aspects of this second embodiment, the pharmaceutical composition further comprises at least one excipient.

A third embodiment of the invention relates to a process for isolating a drug substance, the process comprising a) precipitating a compound of structural formula I:

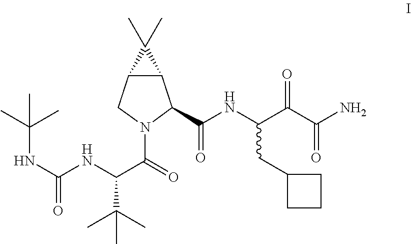 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

at a temperature below about 5.0° C. from a supersaturated solution to form a slurry; b) optionally distilling the slurry to form a concentrate; c) filtering the concentrate to form a wet cake; and d) drying the wet cake to form a powder; wherein the powder comprises isolated drug substance; and the isolated drug substance has a BET specific surface area of from about 2.9 m²/g to about 94 m²/g.

In a first aspect of this third embodiment, the drug substance has a BET specific surface area of from about 2.9 m²/g to about 9.6 m²/g. In specific instances of this first aspect of this third embodiment, the drug substance has a BET specific surface area of from about 2.9 m²/g to about 9.4 m²/g. In all instances of this first aspect, all steps are as provided in any or all other aspects of the third embodiment.

In a second aspect of the third embodiment, the distilling step b) is conducted. In all instances of this second aspect, all steps are as provided in any or all other aspects of the third embodiment.

In a third aspect of the third embodiment, the distilling step b) is conducted at a temperature in a range from about −15.0° C. to about 35.0° C. In some instances of the third aspect of the third embodiment, the distilling step b) is conducted at a temperature in a range from about 15.0° C. to about 30.1° C., such as in a range from about 15.1° C. to about 24.6° C. In specific instances, the distilling step b) is conducted at a temperature in a range from about 15.0° C. to about 30.1° C., such as in a range from about 15.1° C. to about 24.6° C., for the first 10 hours of distillation. In some additional instances of the third aspect of the third embodiment, the distilling step b) is conducted at a temperature in a range from about −15.0° C. to about 15.0° C. In all instances of this third aspect, all other steps are as provided in any or all other aspects of the third embodiment.

In a fourth aspect of the third embodiment, the distilling step b) is conducted over 20 to 30 hours, such as in about 24 hours. In all instances of this fourth aspect, all other steps are as provided in any or all other aspects of the third embodiment.

In a fifth aspect of the third embodiment, the filtering step c) is conducted at a temperatures in a range from of about −20.0° C. to about 15.0° C., such as in a range of from about −15.0° C. to about 15.0° C. In all instances of this fifth aspect, all other steps are as provided in any or all other aspects of the third embodiment.

A fourth embodiment of the invention relates to a process for isolating a drug substance comprising a) precipitating compounds of structural formula I:

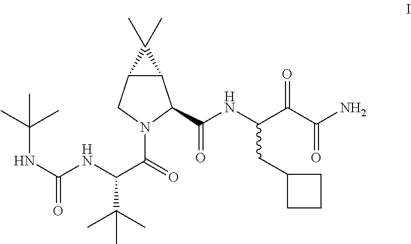 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

at a temperature below about 5.0° C. from a supersaturated solution to form a slurry; b) heating the slurry to an aging temperature of between 5.0° C. and 25° C. and holding the slurry at the aging temperature for a period of time; c) distilling the slurry to form a concentrate, at a temperature of between about −5.0° C. to about 35.0° C., where the distilling temperature is equal or lower to the aging temperature, for the first 4-6 hours of distillation; d) filtering the concentrate to form a wet cake; and e) drying the wet cake to form a powder; wherein the powder comprises the isolated drug substance; and the isolated drug substance has a BET specific surface area of from about 2.93 m²/g to about 94 m²/g.

In a first aspect of this fourth embodiment, the drug substance has a BET specific surface area of from about 2.9 m²/g to about 9.6 m²/g. In specific instances of this first aspect of this fourth embodiment, the drug substance has a BET specific surface area of from about 2.9 m²/g to about 9.4 m²/g. In all instances of this first aspect, all steps are as provided in any or all other aspects of the fourth embodiment.

In a second aspect of the fourth embodiment, the heating step b) is conducted at a temperature in a range from about 14° C. to about 18° C. In all instances of this second aspect, all other steps are as provided in any or all other aspects of the fourth embodiment.

In a third aspect of the fourth embodiment, the slurry of heating step b) is held at the aging temperature for up to 16 hours. In particular instances, the slurry is held at the aging temperature for 6 hours. In all instances of this third aspect, all other steps are as provided in any or all other aspects of the fourth embodiment.

In a fourth aspect of the fourth embodiment, the distilling step c) is conducted at a temperature in a range from about 0.0° C. to about 35.0° C. In some instances of the fourth aspect of the fourth embodiment, the distilling step c) is conducted at a temperature in a range from about 13.0° C. to about 30.1° C. In all instances of this fourth aspect, all other steps are as provided in any or all other aspects of the fourth embodiment.

In a fifth aspect of the fourth embodiment, the distilling step c) is conducted at a temperature that is equal or lower than the aging temperature for the first 4 to 6 hours of distilling. In all instances of this fifth aspect, all other steps are as provided in any or all other aspects of the fourth embodiment.

In a sixth aspect of the fourth embodiment, the distilling step c) is conducted over 20 to 30 hours. In all instances of this sixth aspect, all other steps are as provided in any or all other aspects of the fourth embodiment.

A fifth embodiment of the invention relates to a drug substance prepared by processes according to the third or fourth embodiments. In aspects of the fifth embodiment, the drug substance has a BET specific surface area of from about 2.9 m²/g to about 9.6 m²/g. In specific aspects of this fifth embodiment, the drug substance has a BET specific surface area of from about 2.9 m²/g to about 9.4 m²/g.

A sixth embodiment of the invention relates to pharmaceutical compositions comprising the drug substance according to the fifth embodiment and a pharmaceutically acceptable carrier. In aspects of the sixth embodiment, the pharmaceutical compositions further comprise at least one excipient.

In a seventh embodiment of the invention, the drug substance of the invention is selected from the exemplary species depicted in the Examples shown below.

An eighth embodiment of the invention relates to pharmaceutical compositions comprising the drug substance according to the seventh embodiment and a pharmaceutically acceptable carrier. In aspects of the eighth embodiment, the pharmaceutical compositions further comprise at least one excipient.

Other embodiments of the present invention include the following:

(a) The pharmaceutical composition of the second, sixth or eighth embodiments, further comprising a second therapeutic agent selected from the group consisting of HCV antiviral agents, immunomodulators, and anti-infective agents.

(b) The pharmaceutical composition of (a), wherein the HCV antiviral agent is an antiviral selected from the group consisting of HCV protease inhibitors and HCV NS5B polymerase inhibitors.

(c) A pharmaceutical combination that is (i) a pharmaceutical composition of the second, sixth or eighth embodiments and (ii) a second therapeutic agent selected from the group consisting of HCV antiviral agents, immunomodulators, and anti-infective agents; wherein the pharmaceutical composition of the second, sixth or eighth embodiments and the second therapeutic agent are each employed in an amount that renders the combination effective for inhibiting HCV NS3 protease, or for treating HCV infection and/or reducing the likelihood or severity of symptoms of HCV infection.

(d) The combination of (c), wherein the HCV antiviral agent is an antiviral selected from the group consisting of HCV protease inhibitors and HCV NS5B polymerase inhibitors.

(e) A method of inhibiting HCV NS3 protease in a subject in need thereof, that comprises administering to the subject an effective amount of a pharmaceutical composition of the second, sixth or eighth embodiments.

(f) A method of treating HCV infection and/or reducing the likelihood or severity of symptoms of HCV infection in a subject in need thereof, that comprises administering to the subject an effective amount of a pharmaceutical composition of the second, sixth or eighth embodiments.

(g) The method of (f), wherein the pharmaceutical composition of the second, sixth or eighth embodiments is administered in combination with an effective amount of at least one second therapeutic agent selected from the group consisting of HCV antiviral agents, immunomodulators, and anti-infective agents.

(h) The method of (g), wherein the HCV antiviral agent is an antiviral selected from the group consisting of HCV protease inhibitors and HCV NS5B polymerase inhibitors.

(i) A method of inhibiting HCV NS3 protease in a subject in need thereof, that comprises administering to the subject the pharmaceutical composition of the second, sixth or eighth embodiments or of the embodiments of (a)-(d).

(j) A method of treating HCV infection and/or reducing the likelihood or severity of symptoms of HCV infection in a subject in need thereof, that comprises administering to the subject the pharmaceutical composition of the second, sixth or eighth embodiments or of the embodiments of (a)-(d).

(k) A use of the drug substance of the first, fifth or seventh embodiments in the prevention or treatment of infection by HCV or in the reduction of likelihood or severity of symptoms of HCV infection of in a subject in need thereof. As used herein, the term “prevention” indicates that use of the drug substance may reduce the severity or likelihood of infection by HCV, and the term “treatment” indicates that use of the drug substance may reduce viral load or severity of symptoms associated with HCV infection.

(l) A use of the pharmaceutical composition of the second, sixth or eighth embodiments or of the embodiments (a) through (d) in the prevention or treatment of infection by HCV or in the reduction of likelihood or severity of symptoms of HCV infection of in a subject in need thereof.

(m) A use of process according to the third or fourth embodiments to make a compound for the prevention or treatment of infection by HCV or in the reduction of likelihood or severity of symptoms of HCV infection of in a subject in need thereof.

(n) A use of process according to the third or fourth embodiments to make a compound for the reduction of likelihood or severity of symptoms of HCV infection of in a subject in need thereof.

The present invention also includes a drug substance of the present invention for use (i) in, (ii) as a medicament for, or (iii) in the preparation of a medicament for: (a) inhibiting HCV NS3 protease, or (b) treating HCV infection and/or reducing the likelihood or severity of symptoms of HCV infection. In these uses, the drug substances of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in embodiments (a) through (n) above and the uses set forth in the preceding paragraph, wherein the compound of the present invention employed therein is a drug substance of one of the embodiments, aspects, classes, sub-classes, or features of the compounds described above.

In the embodiments provided herein, it is to be understood that each embodiment may be combined with one or more other embodiments, to the extent that such a combination provides a stable drug substance and is consistent with the description of the embodiments. It is further to be understood that the embodiments of compositions and methods provided as embodiments (a) through (n) above are understood to include all embodiments of the drug substances, including such embodiments as result from combinations of embodiments.

In order to achieve advantageous intermediate and finished product attributes for solid dosage forms of drug substances, such as boceprevir, formulation process robustness studies are often performed to assess potential effects of impeller speed, rate of granulating solution addition, wet massing time, water quantity, and drug substance BET specific surface area. In addition, the effects of low BET specific surface area on formulation processability are separately studied and used to identify a value for the lowest BET specific surface area for a drug substance that provides desirable drug product attributes, such as dissolution. For boceprevir, such studies are exemplified as Examples 6-8.

Through these tests, it was established that drug substance Brunnauer-Emmett-Teller specific surface area (“BET specific surface area” or “BET SSA”) in a particular range is desirable to obtain desirable dissolution properties in the final product. BET specific surface area can be measured by physical adsorption methods known to one of skill in the art. All measurements described herein were made on a TRISTAR 2000 instrument (MICROMERITICS, Norcross, Ga.) using the nitrogen adsorption method, as described in Webb, P. A., “Surface Area, Porosity, and Related Physical Characteristics,” in PHARMACEUTICAL DOSAGE FORMS: TABLETS, VOLUME 3: MANUFACTURE AND PROCESS CONTROL 277-302 (eds. L. Augsburger & S. W. Hoag Informa Healthcare USA Inc. 2008). In the nitrogen absorption method, a tube containing a solid sample is preconditioned at a certain temperature for a predetermined amount of time to remove gases/vapors adsorbed on the surface of the solids. For the examples described herein, the equilibration temperature was 30° C., and the time was 16 hours. The tube is then placed into the instrument and evacuated. After evacuation, the tube is submerged into liquid nitrogen, and a known amount of nitrogen (N₂) gas is introduced in the tube by means of a control valve. The N₂ gas expands and fills the free volume of the tube. The pressure P of the tube will become equal to nRT/V, where n is the number of N₂ gas molecules introduced in the sample tube, R is the universal gas constant, T is the absolute temperature and V is the tube free volume. By repeating the evacuation and filling procedure for different amounts of N₂ gas, a BET isotherm curve can be obtained, and the BET specific surface area of the solids can be calculated from the slope of the BET curve, as described in Webb (supra).

In the process for isolating a drug substance of the claims, the process comprises four primary steps, precipitation, distillation, filtration and drying. Specifically, a process for isolating a drug substance comprises:

a) precipitating a compound of structural formula I:

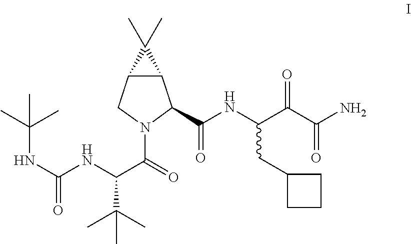 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

at a temperature below about 5.0° C. from a supersaturated solution to form a slurry;

b) optionally distilling said slurry to form a concentrate;

c) filtering the slurry or concentrate to form a wet cake; and

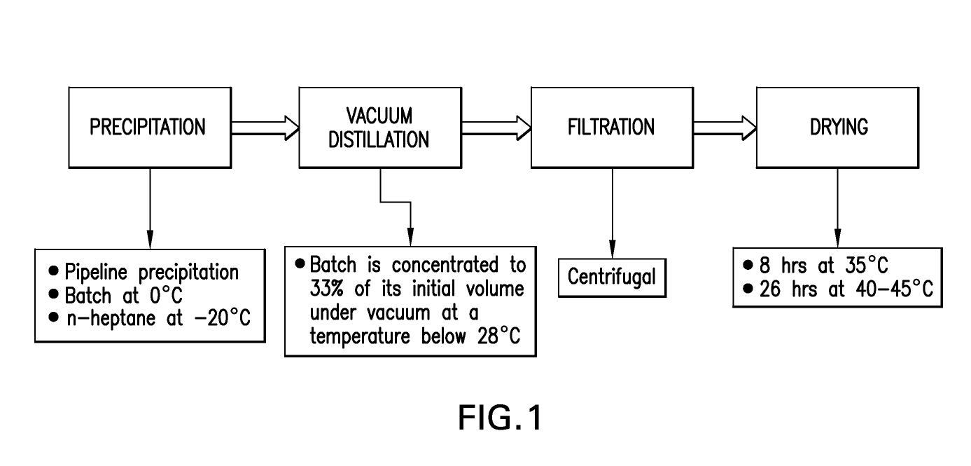

d) drying the wet cake to form a powder;

wherein the powder comprises isolated drug substance; and the isolated drug substance has a BET specific surface area of from about 2.9 m²/g to about 94 m²/g. This isolation process is as shown schematically in FIG. 1.

The precipitation step is the only step involving particle formation. Precipitation conditions affect only the initial value of each drug substance physical attribute. Drug substance attributes may continue to change throughout the rest of the process due to the amorphous nature of the drug substance.

The particle formation in the precipitation step defines the initial drug substance BET specific surface area, the appearance of the solids formed and the polymorphic form. Like other precipitation processes, it is affected by two factors: supersaturation and mixing intensity. Supersaturation is controlled by the composition of the stream. Mixing intensity is controlled by the geometry of the mixing chamber used to mix the batch and the anti-solvent, such as n-heptane, the anti-solvent:batch volumetric ratio (or equivalently the velocity ratio) and on a second level by the anti-solvent Reynolds number. International Patent Application Publication No. WO2007/127380(A2), U.S. Patent Application Publication No. US2008/0193518 and U.S. Patent Application Publication No. US2008/0254128, the disclosures of each of which are incorporated herein by reference, describe details relating to these effects. The precipitation results in drug substance with extremely high surface areas, which are shown in Table 1 for commercial scale tee mixer geometries and the conditions described in Example 3. Even higher surface areas, between 69 m²/g and 94 m²/g, have been produced in laboratory scale tee mixers as described in Example 2.

TABLE 1
Drug Substance BET SSA After Precipitation.

| Batch | BET SSA (m²/g) |
|---|---|
| 1 | 38.08 |
| 2 | 44.01 |
| 3 | 52.40 |
| 4 | 42.07 |
| 5 | 41.61 |
| 6 | 47.40 |
| 7 | 59.56 |
| 8 | 50.28 |
| 9 | 50.41 |
| 10 | 44.24 |
| 11 | 50.62 |
| 12 | 40.41 |
| 13 | 54.93 |
| 14 | 16.59 |

The high drug substance BET specific surface area formed during precipitation may remain unaltered or dramatically change during subsequent processing due to the amorphous nature of the drug substance. Amorphous compounds or materials are characterized by a characteristic temperature, the glass transition temperature. Below the glass transition temperature, amorphous materials behave like glasses; no or very limited movement of the molecules occurs in the solid state. However, above the glass transition temperature, the molecules of the amorphous substance acquire mobility. At sufficiently high temperatures flow phenomena have been observed. In addition, the time that an amorphous drug substance spends above the glass transition temperature affects its properties. The glass transition temperature depends on the composition of the system; solvents that dissolve the drug substance, like MTBE and acetic acid, significantly depress the glass transition temperature of the drug substance. The same is true for water, a known plasticizer of pharmaceutical molecules.

The distilling step of this process can be accomplished by batch distilling a batch volume in a batch vessel. The distillation step includes the heating of the solvent to reflux temperature and the concentration of the batch from 30X to 10X under vacuum, where X is the batch size in kg. During this step, the batch temperature and composition change in a dynamic way. Most changes in drug substance BET specific surface area occur during this step.

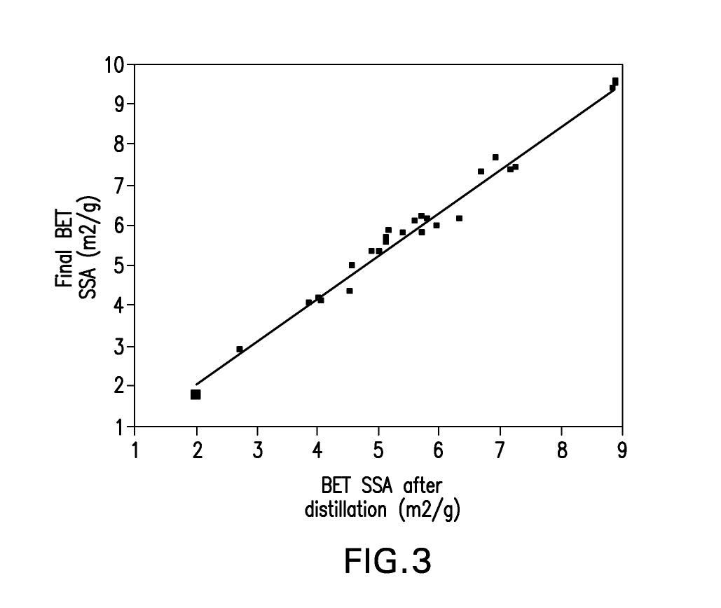

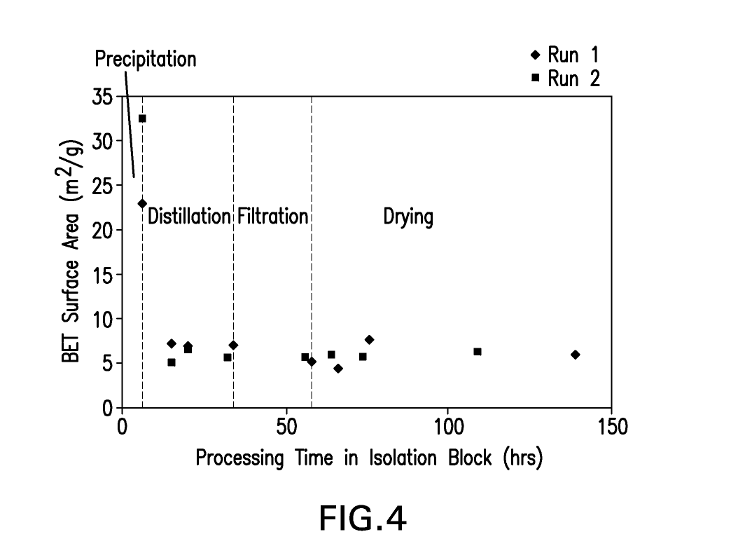

During the distillation step, both temperature and composition change over time. Therefore, the distillation step is expected to define the drug substance BET specific surface area. This expectation materialized as shown in FIG. 3, which illustrates a strong linear correlation (as shown by the correlation coefficient, R²=0.99) between the final drug substance BET specific surface area and the BET specific surface area after the distillation step. As has been demonstrated previously and disclosed in International Patent Application Publication No. WO2007/127380(A2), U.S. Patent Application Publication No. US2008/0193518 and U.S. Patent Application Publication No. US2008/0254128, only the first 9 to 10 hours of distillation are critical for defining the drug substance BET specific surface area as shown in FIG. 4.

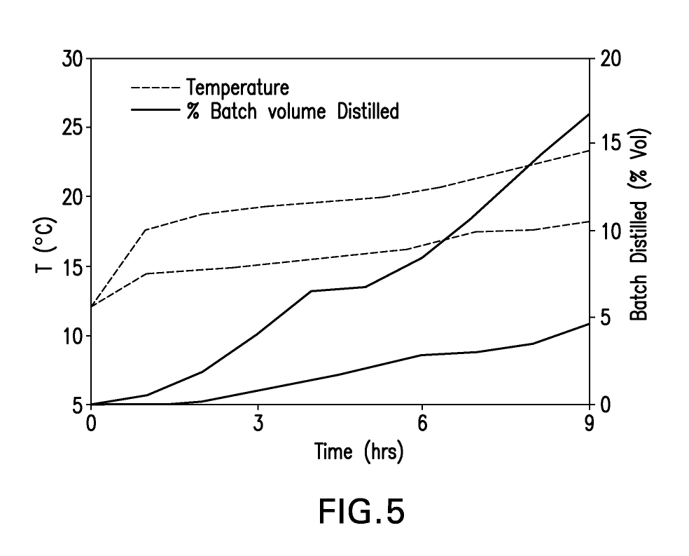

During the distillation step, the batch temperature and composition must be controlled within narrow limits. It is known to those skilled in the art, that for batch distillations the composition can be lumped into a single parameter, the % batch volume distilled, provided the initial composition of the batch is controlled within narrow levels. A careful analysis of batch data for a number of batches executed according to the conditions described in Example 3 showed that drug substance having a BET specific surface area in a desirable range was obtained when the batch temperature and % batch volume distilled were within the ranges described in Table 2. Table 2 displays a set of batch temperatures and % batch volume distilled for each of three time points within the first ten hours of distillation; in Table 2, “onset of distillation” occurs when the batch temperature exceeds 12.1° C. while heating from −15.0° C. FIG. 5 is a graphical representation of the information provided in Table 2 and in Example 3. All of these batches resulted in drug substance having BET specific surface area within a range of 2.9 m²/g to 9.6 m²/g. It was further found that an overall maximum batch temperature of 30.1° C. resulted in this range. Thus, it was surprisingly found that batch distillation temperatures and the rate of distillation during the first ten hours of distillation impact drug substance BET specific surface area.

TABLE 2
Distillation profile.

| Time from onset | Batch Temperature | % Batch Volume |
|---|---|---|
| of distillation (h) | (° C.) | Distilled |
| 3 | 15.1 to 22.5 | 0.0 to 2.8 |
| | 17.7 to 22.5 | 2.8 to 4.1 |
| 6 | 16.3 to 22.4 | 2.8 to 7.0 |
| | 19.1 to 22.4 | 7.0 to 8.4 |
| 10 | 19.2 to 24.2 | 5.9 to 11.4 |
| | 19.2 to 24.6 | 11.4 to 14.7 |
| | 20.2 to 24.6 | 14.7 to 18.8 |

This description is intended to be illustrative and not limiting. Various changes or modifications in the embodiments described herein may occur to those skilled in the art. For example, it was shown that at low temperatures (such as −15.0° C. or lower), the drug substance BET specific surface area remains unchanged. One skilled in the art could design a cold filtration process that would retain the BET specific surface area value as high as the one delivered by the precipitation step. Alternatively, one could perform the distillation step at very low temperatures (from below about −10.0° C. to −15.0° C.) if the appropriate equipment is available. Therefore, it is possible to isolate drug substance with BET specific surface area as high as 60 m²/g if a commercial scale tee mixer is used or even 94 m²/g if a laboratory scale tee mixer is used. These changes can be made without departing from the scope of spirit of the invention.

During the filtration step, the composition of the batch stream remains unchanged; only processing temperature and time vary during this step. The product of the filtering step of the process described in Example 3 is in the form of a wet cake, which contains only small amounts of solvents, such as water, acetic acid (AcOH) and methyl tert-butyl ether (MTBE), that depress the glass transition temperature of the drug substance. The glass transition temperature is much higher than the filtration temperature; consequently, changes in the drug substance BET specific surface area were not observed. FIG. 4 shows this for two commercial scale batches prepared by the processes according to Example 3. Moreover, prolonged holding of the wet cake does not affect the drug substance BET specific surface area. Therefore, the drug substance BET specific surface area is not affected during this step.

In different embodiments of this invention, such as those in which distillation is not performed, the MTBE, acetic acid and water content may be considerably higher compared to the process described in Example 3 and the glass transition temperature may be below 10° C.-20° C. In such embodiments, the filtration temperature may affect the drug substance BET specific surface area. Temperatures below the glass transition will minimize or eliminate the BET specific surface area reduction. Thus, drug substance BET specific surface area may be controlled to desired levels by manipulating filtration temperature.

In the drying step, solvent is removed. However, the levels of solvents, such as water, acetic acid (AcOH) and methyl tert-butyl ether (MTBE), during this step are sufficiently low to ensure a high drug substance glass transition temperature and therefore minimize any changes in drug substance BET specific surface area. The latter is affected only by attrition, which takes place exclusively at the beginning of the drying process but does not significantly affect the drug substance BET specific surface area as shown in FIG. 4 for two commercial scale batches prepared according to the methods described in Example 3.

An alternative isolation process, otherwise referred to as an “aging” isolation process, for isolating a drug substance comprises

a) precipitating compounds of structural formula I:

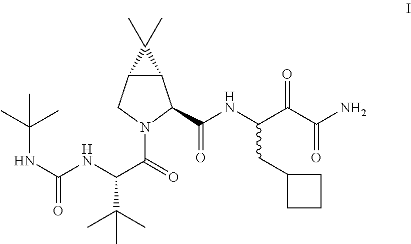 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

at a temperature below about 5.0° C. from a supersaturated solution to form a slurry;

b) heating the slurry to an aging temperature of between 5.0° C. and 25° C. and holding the slurry at the aging temperature for a period of time;

c) distilling the slurry to form a concentrate, at a temperature of between about −5.0° C. to about 35.0° C., where the distilling temperature is equal or lower to the aging temperature, for the first 4 to 6 hours of distillation;

d) filtering the concentrate to form a wet cake; and

e) drying the wet cake to form a powder; wherein the powder comprises the isolated drug substance; and the isolated drug substance has a BET specific surface area of from about 2.9 m²/g to about 94 m²/g.

As in the previously described process, the distilling step of this process can be accomplished by batch distilling a batch volume in a batch vessel, and the product of the filtering step may be in the form of a wet cake.

During precipitation, the temperature is maintained at temperatures below 5.0° C.

The aging process can deliver drug substance at a desired BET specific surface area range of from about 2.9 m²/g to about 9.6 m²/g, such as from about 2.9 m²/g to about 9.4 m²/g. Lower aging temperatures will result in drug substance with higher BET specific surface areas. Higher aging temperatures will result in even lower drug substance BET specific surface areas, which are not suitable for formulation in accordance with desired parameters. In the above process, BET specific surface area control is achieved with the aging step. The distillation step simply serves as the step that “freezes” the drug substance BET specific surface area to the value achieved during the aging step. In this role, the distillation process does not have to follow the elaborate batch temperature-% batch volume distilled profile described in Table 2.

### EXAMPLES

### Abbreviations

AcOH Acetic acid

C, ° C. Temperature in Celsius

h Hours

HCl Hydrochloric acid

KBr Potassium bromide

kg Kilograms

Kg/min/kg Kg per minute of solution per kg of solids

L Liters

M Molar

m²/g Meters squared per gram

min Minutes

mL Milliliters

MTBE Methyl tert-butyl ether

N Normal

N₂ Nitrogen gas

NaOAc Sodium acetate

NaOCl Sodium hypochlorite

RPM Revolutions per minute

RT Room temperature, approximately 25° C.

T Temperature

TEMPO 2,2,6,6-tetramethyl-1-piperidinyloxy free radical (Aldrich)

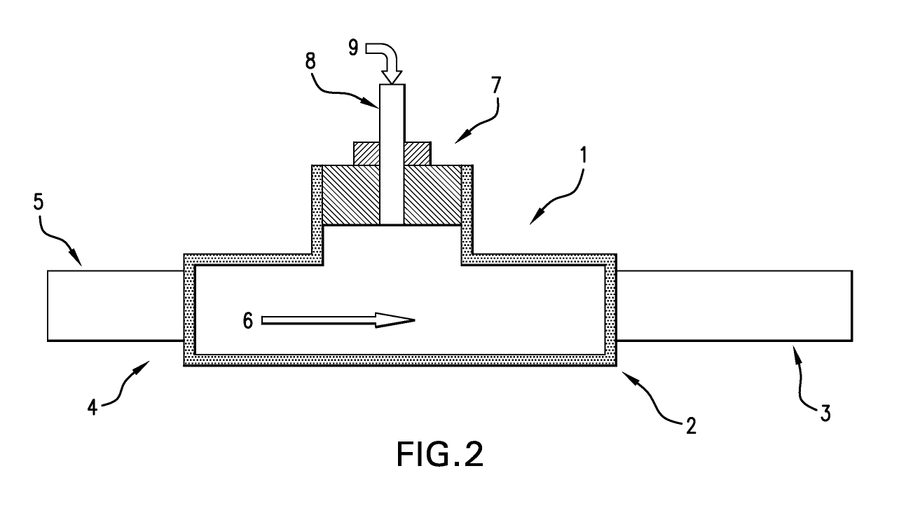

Mixing chambers were constructed according to FIG. 2. The mixing chamber comprises a mixing tee (1), and optionally connected to the outlet leg (2) of the tee run, static mixer (3), wherein a stream of anti-solvent is passed through the straight run inlet (4) via anti-solvent inlet line (5) in the direction of arrow (6), and a stream of a solution is passed into the branch run (7) via solution inlet line (8) in the direction of arrow (9). In the Examples below, dimensions for diameters of legs and lines of mixing chambers according to FIG. 2 are inside diameters unless otherwise noted.

### Example 1

### Synthesis of Boceprevir

### Procedure A1

A precursor of the compound of structural formula I, boceprevir, was prepared according to the procedure of Example 1 of U.S. Patent Application No. 61/482,592, the disclosures of which are herein incorporated by reference.

### Procedure A2

The compound of structural formula I, boceprevir, was prepared according to the procedure of Example 3 of U.S. Patent Application No. 61/482,592, the disclosures of which are herein incorporated by reference.

### Example 2

A mixing chamber was prepared according to FIG. 2, having a 0.25″ outer diameter batch outlet leg (2), 0.12″ diameter straight run inlet (4) via 0.12″ diameter anti-solvent inlet line (5), and 0.026″ diameter solution inlet line (8).

The product of Example 1, Procedure A2 (198.5 g) was added to MTBE (881.3 g) to prepare a 0.29M solution. Water and AcOH were added to the solution such that the final volume contained 26.8 g of water (26.8 g) and 1.17 g of AcOH. A slurry was prepared by mixing 2,400 mL/min of n-heptane, held at a temperature of 25° C., and 625 mL/min of the 0.29M solution, held at 5° C., in the mixing chamber. The output of the mixing chamber was collected for about 2 to 3 minutes in a stirred holding tank fitted with a temperature-controlled jacket and an agitating paddle and held at RT. The slurry was filtered immediately with a Buchner funnel pre-cooled by contacting it with n-heptane (about −20° C.), dried at temperatures below about 35-45° C. and sampled for BET specific surface area analysis. The drug substance BET specific surface area was measured at about 94 m²/g.

### Example 3

A mixing chamber was prepared according to FIG. 2, having a 1″ diameter batch outlet leg (2), 0.834″ diameter straight run inlet (4) via 0.834″ diameter anti-solvent inlet line (5), and 0.12″ diameter solution inlet line (8).

For each of 14 batches, a solution comprising the product of Example 1, Procedure A2, in concentrations in a range from about 0.25M to 0.32M, was prepared according to the procedures of Example 3 of U.S. Patent Application No. 61/482,592. A slurry was prepared by mixing 20,000 mL/min of n-heptane, held at a temperature of −15° C., and 5,000 mL/min of the solution, held at 5° C., in the mixing chamber. The output of the mixing chamber was collected for about 6.0 to 6.5 hours in a stirred holding tank fitted with a temperature-controlled jacket, vacuum line and an agitating paddle and held below −10° C. Subsequently, the batch was warmed by running the jacket temperature at 15° C. When the slurry attained a temperature of 12.1° C., the vessel was evacuated, and distillation was begun.

During distillation, the pressure and jacket temperature were manipulated to follow the batch temperature and percent batch volume as shown in Table 3. Distillation was continued until the slurry attained a volume that was 33.33% of the initially collected slurry volume.

TABLE 3
Distillation profile.

| Time from onset of | Distillation | % Batch |
|---|---|---|
| distillation (h) | Temperature (° C.) | Volume Distilled |
| 3 | 15.1 to 22.5 | 0.0 to 2.8 |
| | 17.7 to 22.5 | 2.8 to 4.1 |
| 6 | 16.3 to 22.4 | 2.8 to 7.0 |
| | 19.1 to 22.4 | 7.0 to 8.4 |
| 10 | 19.2 to 24.2 | 5.9 to 11.4 |
| | 19.2 to 24.6 | 11.4 to 14.7 |
| | 20.2 to 24.6 | 14.7 to 18.8 |

After distillation, the batches were cooled to a temperature between 0° C. and 10° C., filtered and washed with about 200-300 L fresh n-heptane. Finally, the batches were dried at temperatures below 48° C. to afford the boceprevir drug substance.

Samples of the slurry were collected immediately after the precipitation and the distillation step, filtered, washed with n-heptane (at −10° C. to −20° C.), dried and subjected to BET specific surface area analysis. The results of this analysis are shown in Table 4. Drug substance BET specific surface area values as high as 60 m²/g were produced from the precipitation step. Following the batch temperature-% batch volume distilled profile shown in Table 4 as well as not exceeding a temperature of 30.1° C. during distillation, the drug substance BET specific surface area obtained was between 3 m²/g and 9.4 m²/g. A slight increase in drug substance BET specific surface area was observed during the filtration and drying steps and the final drug substance BET specific surface area ranged between 2.9 m²/g and 9.6 m²/g.

TABLE 4
Evolution of Drug Substance BET SSA During the Isolation Process.

| | BET SSA After | BET SSA After | Maximum | |
|---|---|---|---|---|
| | Precipitation | Distillation | Distillation | Final BET |
| Batch | (m²/g) | (m²/g) | Temperature (° C.) | SSA (m²/g) |
| 1 | 38.08 | 4.88 | 26.1 | 5.36 |
| 2 | 44.01 | 5.58 | 25.6 | 6.10 |
| 3 | 52.40 | 5.71 | 26.4 | 6.22 |
| 4 | 42.07 | 3.99 | 27.6 | 4.22 |
| 5 | 41.61 | 3.85 | 27.5 | 4.07 |
| 6 | 47.40 | 5.12 | 25.8 | 5.56 |
| 7 | 59.56 | 5.11 | 26.3 | 5.73 |
| 8 | 50.28 | 8.86 | 21.8 | 9.59 |
| 9 | 50.41 | 8.84 | 24.5 | 9.41 |
| 10 | N/A | 2.66 | 30.1 | 2.93 |
| 11 | 50.62 | 5.39 | 25.9 | 5.80 |
| 12 | 40.41 | N/A | 26.3 | 5.22 |
| 13 | 54.93 | N/A | 26.5 | 5.02 |
| 14 | 16.59 | 5.96 | 26.8 | 6.00 |

### Example 4

A mixing chamber was prepared according to FIG. 2, having a 1″ diameter batch outlet leg (2), 1″ diameter straight run inlet (4) via 0.834″ diameter anti-solvent inlet line (5), and 0.12″ diameter solution inlet line (8).

The product of Example 1, Procedure A1 (320 kg), KBr (64 kg), NaOAc (64 kg), TEMPO (96 kg), glacial AcOH (234 kg) and MTBE (2560 L) were charged in a 11000 L reactor equipped with a retreat curve impeller temperature probes and a temperature control jacket. The mixture was cooled to a temperature between 10° C. and 20° C. A solution of 5% NaOCl (about 1100 L) was added to the mixture over 2 h to 3 h while the temperature was maintained between 10° C. and 20° C. After NaOCl addition, the mixture was agitated for 3 h. Water (320 L) was then added; the temperature of the mixture adjusted to between 0° C. and 10° C.; and the organic and aqueous layers were separated. The batch was then washed one more time with water (1600 L). A solution of ascorbic acid, prepared from sodium ascorbate (320 kg), 36% HCl solution (166 kg) and water (1450 L), was added to the batch over about 2 h, while the temperature was maintained between 5° C. and 10° C. The mixture was agitated for 3 h, and the two layers were separated. Subsequently, the batch was washed with a 3.5N HCl solution, prepared from water (900 L) and 36% HCl solution (454 kg), while maintaining the temperature between 0° C. and 10° C. The two layers were then separated, and the organic layer was washed four times with water (1600 L) at a temperature between 0° C. and 10° C. The batch volume was then adjusted to about 1920 L.

A slurry was prepared by mixing 20,000 mL/min of n-heptane, held at a temperature of −20° C., and 5,000 mL/min of the batch, held at 0° C., in the mixing chamber. The output of the mixing chamber was collected for about 6.0 h to 6.5 h in a stirred holding tank fitted with a temperature-controlled jacket, vacuum line and an agitating paddle and held below −10° C. Subsequently, the batch was warmed to an “aging” temperature of 15° C. and equilibrated at this temperature for 6 h. Once 6 h elapsed, the vessel was evacuated, and distillation was begun at reflux temperatures between 13° C. and 15° C. The vessel was evacuated to achieve full vacuum, to drive distillation as quickly as possible. Distillation was continued at temperatures below 23.1° C. until the slurry attained a volume that was 33.33% of the initially collected slurry volume.

After distillation, the batch was cooled to a temperature between 0° C. and 10° C., filtered and washed with about 200-300 L fresh n-heptane. Finally, the batch was dried at temperatures below 48° C. to afford the boceprevir drug substance. Samples of the slurry were collected immediately after the precipitation and the distillation step, filtered, washed with n-heptane (at −10° C. to −20° C.), dried and subjected to BET specific surface area analysis. The results are shown in Table 5. Excellent batch-to-batch reproducibility was obtained.

TABLE 5
Distillation profile.

| | | BET SSA | BET SSA | Maximum | |
|---|---|---|---|---|---|
| | Aging | After | After | Distillation | Final |
| | Temperature | Precipitation | Distillation | Temperature | BET SSA |
| Batch | (° C.) | (m²/g) | (m²/g) | (° C.) | (m²/g) |
| 1 | 14 | 56.70 | 8.88 | 26.1 | 9.30 |
| 2 | 15 | 46.94 | 6.92 | 25.6 | 7.61 |
| 3 | 15 | 53.85 | 7.22 | 26.4 | 7.47 |
| 4 | 15 | 47.48 | 6.67 | 27.6 | 7.31 |
| 5 | 15 | 48.30 | 7.16 | 27.5 | 7.41 |

### Example 5

### Dissolution and High-Shear, Wet-Granulation Studies

Boceprevir drug substance having BET specific surface areas in a range of 4.19 m²/g and 9.41 m²/g, prepared according to Example 3, was used to manufacture twenty formulation batches of drug product with different high-shear granulation conditions in a half-fractional factorial, statistically designed set of experiments, and was evaluated to determine formulation process robustness. This robustness study was performed for the high-shear, wet-granulation formulation process used to manufacture the boceprevir drug product. Several processing parameters such as impeller speed, rate of granulating solution addition, wet-massing time, amount of water added and drug substance loading were examined in a half-fractional factorial statistically designed set of experiments, with four replicate center points. These experiments were conducted using a 150 L high-shear granulator and using a batch size of 31.25 kg. The statistical design and the granulation parameters examined are summarized in Table 6.

TABLE 6
Drug Product Dissolution Process Parameters.

| | Impeller | Spray | Wet | Water | Drug |
|---|---|---|---|---|---|
| Randomized | Speed | Rate | Massing | Quantity | Substance |
| Run Order | (rpm) | (g/min/kg) | Time (min) | (kg) | SSA (m²/g) |
| 1 | 122 | 0.052 | 5 | 21.25 | 4.19 |
| 2 | 97 | 0.052 | 1 | 21.25 | 9.41 |
| 3 | 110 | 0.060 | 3 | 20 | 6.18 |
| 4 | 122 | 0.068 | 1 | 19.25 | 4.19 |
| 5 | 97 | 0.052 | 5 | 21.25 | 9.41 |
| 6 | 122 | 0.052 | 5 | 19.25 | 4.19 |
| 7 | 122 | 0.052 | 1 | 19.25 | 9.41 |
| 8 | 110 | 0.060 | 3 | 20 | 6.18 |
| 9 | 122 | 0.068 | 1 | 21.25 | 9.41 |
| 10 | 110 | 0.060 | 3 | 20 | 6.18 |
| 11 | 122 | 0.052 | 5 | 19.25 | 9.41 |
| 12 | 97 | 0.052 | 1 | 19.25 | 4.19 |
| 13 | 97 | 0.068 | 5 | 21.25 | 4.19 |
| 14 | 110 | 0.060 | 3 | 20 | 6.18 |
| 15 | 122 | 0.068 | 1 | 21.25 | 4.19 |
| 16 | 97 | 0.052 | 1 | 21.25 | 4.19 |
| 17 | 97 | 0.068 | 5 | 19.25 | 4.19 |
| 18 | 122 | 0.068 | 5 | 21.25 | 9.41 |
| 19 | 97 | 0.068 | 5 | 19.25 | 9.41 |
| 20 | 97 | 0.068 | 1 | 19.25 | 9.41 |

The resulting drug products were subjected to dissolution testing in a USP Dissolution Apparatus 2 (DISTEK, New Brunswick, N.J.), equipped with a paddle (see United States Pharmacopeia (USP) General Chapter 711 Dissolution). 50 millimoles of phosphate buffer (pH=6.8) containing 0.1% sodium dodecyl sulphate was used. Testing was performed at physiological temperatures (37.0° C.±0.5° C.). The results of the dissolution testing can be shown in Table 7.

TABLE 7
Dissolution Performance.

| | BET SSA | % Dissolved at 45 min |
|---|---|---|
| Batch | (m²/g) | (% released, average n = 6) |
| 1 | 4.19 | 95.4 |
| 2 | 9.41 | 90.5 |
| 3 | 6.18 | 97.1 |
| 4 | 4.19 | 98.2 |
| 5 | 9.41 | 83.3 |
| 6 | 4.19 | 93.7 |
| 7 | 9.41 | 89.5 |
| 8 | 6.18 | 92.3 |
| 9 | 9.41 | 83 |
| 10 | 6.18 | 93.1 |
| 11 | 9.41 | 78.1 |
| 12 | 4.19 | 95.7 |
| 13 | 4.19 | 97.3 |
| 14 | 6.18 | 93.6 |
| 15 | 4.19 | 99.2 |
| 16 | 4.19 | 98.3 |
| 17 | 4.19 | 94.1 |
| 18 | 9.41 | 87.4 |
| 19 | 9.41 | 81.4 |
| 20 | 9.41 | 86.4 |

As can be seen from Table 7, after 45 min, all twenty test batches met or exceeded a 75% dissolution criterion.

A robustness study was performed for the high-shear, wet-granulation formulation process used to manufacture the boceprevir drug product. Several processing parameters such as impeller speed, rate of granulating solution addition, wet-massing time, amount of water added and drug substance loading were examined. The ranges of these process parameters examined are given in Table 8 below. Drug product batches were conducted using a 150 L high-shear granulator, using a batch size of 31.25 kg. The results were analyzed by statistical methods to determine which factor(s) influence(s) finished product dissolution. A summary of the statistical analysis is given in Table 8 below, which shows the p-values for each factor studied. A p-value smaller than 0.05 indicate a factor that is statistically significant at a 95% confidence level and can be considered influential to drug product dissolution. The results of Table 7 clearly illustrate that only the drug substance BET specific surface area directly influenced the dissolution rate of the finished product at a statistically significant level.

TABLE 8
Drug Product Dissolution Process Robustness.

| Factor | Range | P-value |
|---|---|---|
| Impeller Speed (RPM) | 97-122 | 0.762 |
| Granulation Solution Addition | 0.052-0.068 | 0.958 |
| Rate (kg/min/kg) | | |
| Wet Massing Time (min) | 1-5 | 0.063 |
| Water Quantity (kg) | 19.25-21-25 | 0.083 |
| Drug Substance BET SSA (m²/g) | 4.19-9.41 | <0.0001 |

As can be seen from this study, only the BET specific surface area has a statistically significant effect on dissolution.

### Example 6

### Low BET Specific Surface Area Study

Boceprevir drug substances having BET specific surface area of 2.93 m²/g, and 2.01 m²/g were prepared according to the process parameters of Example 3.

2.93 m²/g BET Specific Surface Area

The drug substance having BET specific surface area of 2.93 m²/g was prepared by following the distillation profile described in Table 3, and a drug product batch was manufactured at a 6.25 kg batch size, using a 30 L high-shear granulator and the parameters in Table 9. The resulting dissolution performance is summarized in Table 10.

TABLE 9
Drug Product Dissolution Process Parameters.

| Factor | Value |
|---|---|
| Impeller Speed (RPM) | 190 |
| Granulation Solution | 0.067 |
| Addition Rate (kg/min/kg) | |
| Wet Massing Time (min) | 3 |
| Water Quantity (kg) | 4.44 |

TABLE 10
Dissolution Performance

| Time (minutes) | % Dissolved |
|---|---|
| 10 | 86 |
| 20 | 95 |
| 30 | 96 |
| 45 | 98 |
| 60 | 98 |

As can be seen from Table 10, boceprevir drug substance having BET specific surface area in of 2.93 m²/g can be expected to meet or exceed a 75% dissolution criterion, and therefore can be expected to have desired quality and processability attributes.

2.01 m²/g BET Specific Surface Area

The drug substance having BET specific surface area of 2.01 m²/g was obtained according to the distillation profile described in Table 11.

TABLE 11
Distillation profile.

| Time from onset | Distillation | % Batch Volume |
|---|---|---|
| of distillation (h) | Temperature (° C.) | Distilled |
| 3 | 25 | 1.70 |
| 6 | 26.3 | 2.73 |
| 10 | 27.0 | 5.94 |

Physical characteristics of the boceprevir drug substance having BET specific surface area of 2.01 m²/g resulted in testing of particles of this drug substance lot being terminated.

### Example 7

### High BET Specific Surface Area Study

A solid dosage form was prepared containing boceprevir drug substance prepared according to the process parameters of Example 6 and having BET specific surface area of 12.06 m²/g. A drug product batch was manufactured at a 6.25 kg batch size, using a 30 L high-shear granulator and the parameters listed in Table 12. Physical characteristics of the boceprevir drug substance having BET specific surface area of 12.06 m²/g were not within desired ranges. However, Table 13 summarizes the dissolution performance of the boceprevir drug substance having BET specific surface area of 12.06 m²/g.

TABLE 12
Drug Product Dissolution Process Parameters.

| Factor | Value |
|---|---|
| Impeller Speed (RPM) | 190 |
| Granulation Solution | 0.067 |
| Addition Rate (kg/min/kg) | |
| Wet Massing Time (min) | 3 |
| Water Quantity (kg) | 4.44 |

TABLE 13
Dissolution Performance

| Time | % Dissolved | | | |
|---|---|---|---|---|
| (minutes) | Sample 1 | Sample 2 | Sample 3 | ample 4 |
| 10 | 46 | 46 | 53 | 28 |
| 20 | 63 | 62 | 72 | 46 |
| 30 | 72 | 71 | 80 | 54 |
| 45 | 79 | 78 | 86 | 61 |
| 60 | 83 | 82 | 90 | 67 |

As can be seen from Table 13, boceprevir drug substance having BET specific surface area in of 12.06 m²/g did not consistently meet or exceed a 75% dissolution criterion at 45 minutes, and therefore cannot be expected to have desired quality and processability attributes.

### Example 8

### Morphology Study

A mixing chamber was prepared according to FIG. 2, having a 0.375″ outer diameter batch outlet leg (2), 0.305″ diameter straight run inlet (4) via 0.305″ diameter anti-solvent inlet line (5), and 0.069″ diameter solution inlet line (8).

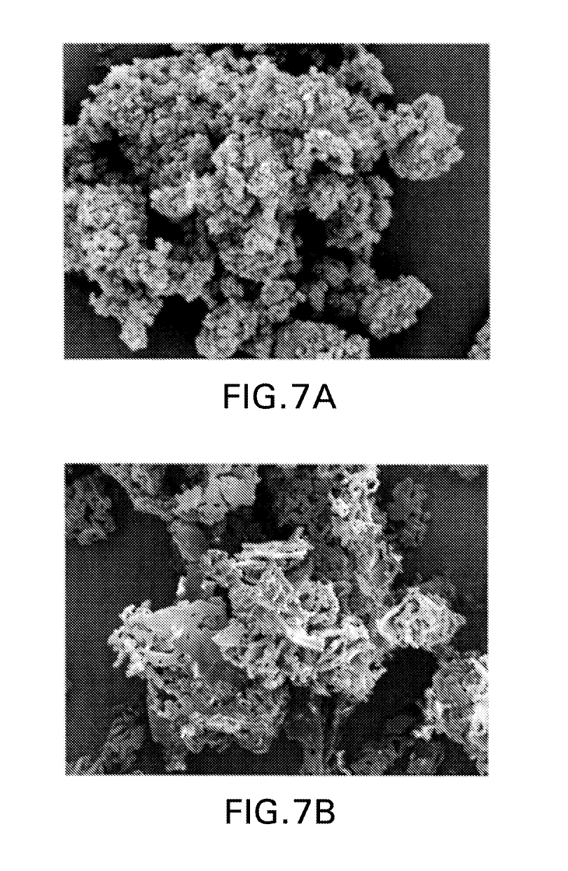

A solution of the product of Example 1, Procedure A2 (608.5 g) dissolved in MTBE (2450 mL) was prepared. The solution was added into the mixing chamber at a rate of 840 mL/min and combined with n-heptane flowing at a rate of 3400 mL/min to form a slurry. The precipitation temperature was controlled at 20° C. After precipitation, one sample was filtered at 20° C. and a second sample was heated to 50° C. at a rate of 1° C./min. The drug substance particle morphology was examined by Scanning Electron Microscopy (SEM); images of the particles are provided as FIGS. 7A and 7B, respectively.

The difference in morphology between the two heated samples is readily apparent. The sample filtered at 20° C., shown in FIG. 7A, retained the particle morphology obtained during precipitation. The sample heated to 50° C., shown in FIG. 7B, consists of particles that are fused together; some particles look melted. The particles having different morphologies also had differences in drug substance physical attributes. Table 14 shows that the powder shown in FIG. 7A has 10 times higher BET specific surface area and about 2 times higher bulk and tap density compared to the powder shown in FIG. 7B.

TABLE 14
Physical Attributes

| Equilibration | | | |
|---|---|---|---|
| Temperature | Bulk Density | Tap Density | BET SSA |
| (° C.) | (g/mL) | (g/mL) | (m²/g) |
| 20 | 0.3087 | 0.4062 | 19.11 |
| 50 | 0.1265 | 0.1898 | 1.82 |

### Example 9

### Surface Area Profile Study

A slurry of product of Example 1, Procedure A2, was prepared by suspending drug substance (25 g, 1 part by mass) in a solvent mixture containing n-heptane (410.2 g, 16.42 parts by mass), MTBE (96.0 g, 3.84 parts by mass) and AcOH (0.147 g, 0.059 parts by mass). The suspension formed was cooled to −15° C. Water (3.374 g, 0.135 parts by mass) was added to precipitate the drug substance and form a slurry, and the slurry was held for 30 min to equilibrate the temperature.

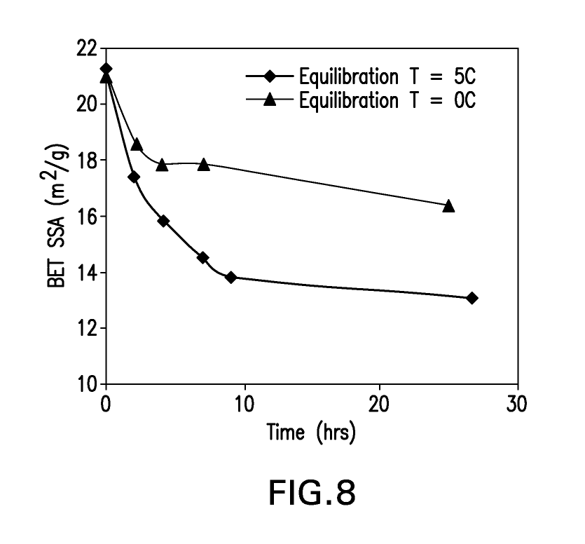

The slurry was held at an initial temperature of −15° C., and split into two parts. The two slurries were then subjected to different constant external temperatures of 5° C. and 0° C., respectively. As slurry temperature increased, a steep decrease in BET specific surface area was observed over time; once a constant temperature was reached, the BET specific surface area continued to decrease at a much slower rate. FIG. 8 shows the boceprevir drug substance BET specific surface area profiles for the slurries of this Example.

FIG. 8 also shows that the final boceprevir drug substance BET specific surface area was lower for the higher equilibration temperature. Therefore, it can be seen that, for the same composition, the rate of BET specific surface area change increases with an increase in operating temperature while the final value of the boceprevir drug substance BET specific surface area decreases with an increase in operating temperature.

### Example 10

### Surface Area Profile Study

A sample of product of Example 1, Procedure A2, in n-heptane was prepared by suspending drug substance (27 g, 1 part by mass) in a solvent mixture containing n-heptane (408.8 g, 15.14 parts by mass), MTBE (1.036 g, 3.84 parts by mass) and AcOH (0.2378 g, 0.088 parts by mass). The suspension formed was cooled to −15° C. Water was added to bring the total amount of water to 3.6429 g (0.135 parts by mass), and the slurry was held for 30 min to equilibrate the temperature at −15° C.

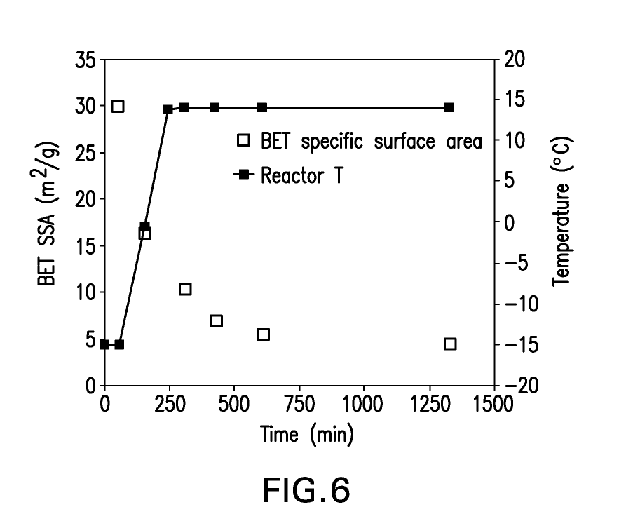

The slurry was heated over 3-4 h to an aging temperature of 14° C. and held at 14° C. until the drug substance BET specific surface area did not change appreciably. FIG. 6 illustrates the change over time of the drug substance BET specific surface area over time, according to this Example. FIG. 6 shows that a decrease in BET specific surface area occurs during heating of the sample from the precipitation to the aging temperature. When the slurry temperature reaches the aging temperature, the decrease in BET specific surface area slows down, and the rate of BET specific surface area reduction becomes smaller as time progresses. It is apparent from FIG. 6 that after 3 hours of holding at the aging temperature, very small changes in BET specific surface area occur, and the dependence of drug substance BET specific surface area on processing time is very weak. Based on this observation, the holding period at the aging temperature was set to 6 hours to ensure minimal variation of drug substance BET specific surface area.

### Example 11

### Surface Area Profile Study

Two slurries were prepared as follows. For Run 1, a slurry of the product of Example 1, Procedure A2, was prepared by suspending drug substance (25.0 g) in a solvent mixture containing n-heptane (410.2 g), MTBE (98.0 g), AcOH (0.145 g) and water (3.371 g), while maintaining the temperature at 5° C. For Run 2, a slurry of the product of Example 1, Procedure A2, was prepared by suspending drug substance (25.0 g) in a solvent mixture containing n-heptane (410.3 g), MTBE (98.0 g), AcOH (0.143 g) and water (3.371 g), while maintaining the temperature at 15° C.

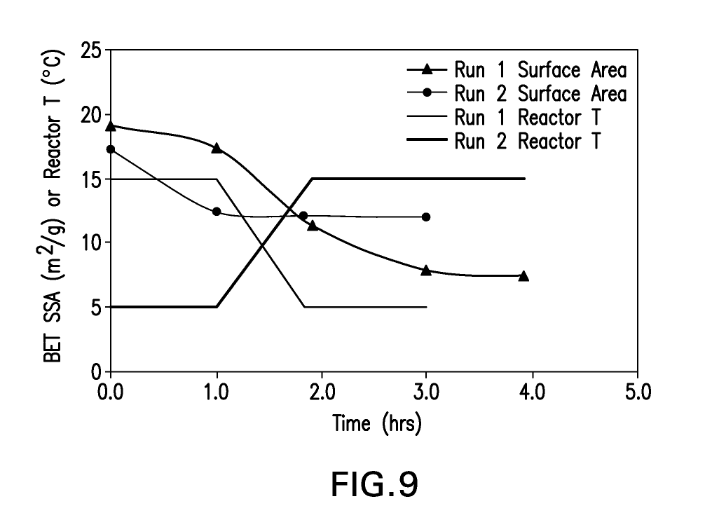

Run 1 was equilibrated for 1 h at 5° C., then heated to 15° C. and kept at that temperature for 2 h. Run 2 was equilibrated for 1 h at 15° C., then cooled to 5° C. and kept at that temperature for 2 h. FIG. 9 illustrates the evolution of drug substance BET specific surface area according to this Example. It can be seen from FIG. 9 that by cooling the slurry, the drug substance BET specific surface area can be locked to a certain value; cooling reduces the difference between the operating temperature and the glass transition temperature of the drug substance and thus locks the rate at which the drug substance agglomerates.

### Example 12

### Composition Study

Two slurries containing samples of the product of Example 1, Procedure A2, were prepared by suspending the same amount of drug substance into the same volume of different solvent mixtures. The first slurry (low solvent/high anti-solvent) was prepared by suspending boceprevir drug substance (22 g, 1 part by mass) into a solvent mixture of MTBE (105.5 g, 4.80 parts by mass), water (4.3532 g, 0.198 parts by mass), AcOH (0.262 g, 0.0119 parts by mass) and n-heptane (397.7 g, 18.1 parts by mass), while maintaining the temperature at −15° C. The second slurry (high solvent/low anti-solvent) was prepared by suspending boceprevir drug substance (30 g, 1 part by mass) into a solvent mixture of MTBE (89.4 g, 2.98 parts by mass), water (1.8921 g, 0.063 parts by mass), AcOH (1771 g, 0.0059 parts by mass) and n-heptane (444.0, 14.8 parts by mass), while maintaining the temperature at −15° C. Both slurries were subjected then to the same constant external temperature (T=18° C.), and the evolution of drug substance BET specific surface area was monitored over time.

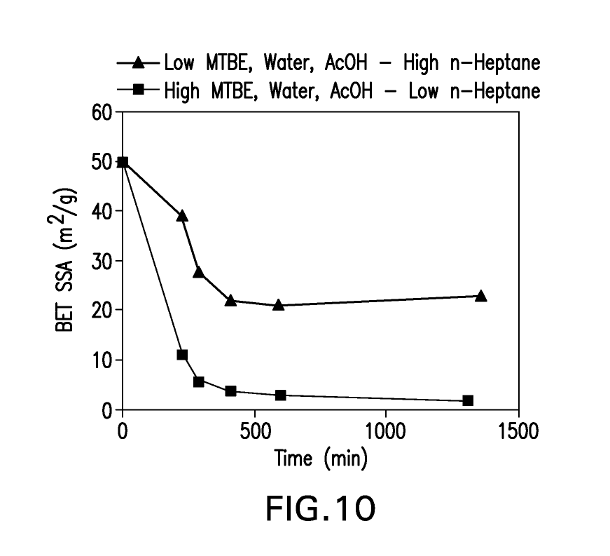

FIG. 10 shows the effect of these changes in slurry composition on the boceprevir drug substance BET specific surface area profiles. As can be seen from FIG. 10, the first slurry results in a higher drug substance BET specific surface area, and the rate of drug substance BET specific surface area change was slower. The second slurry results in a lower drug substance BET specific surface area, and the rate of drug substance BET specific surface area change was faster.

### Example 13

### Aging Study

Five batches of drug substance were prepared according to Example 4. The drug substance BET specific surface area results are summarized in Table 15.

TABLE 15
Distillation profile.

| Batch | Aging Temperature (° C.) | BET SSA (m²/g) |
|---|---|---|
| 1 | 14 | 9.30 |
| 2 | 15 | 7.61 |
| 3 | 15 | 7.47 |
| 4 | 15 | 7.31 |
| 5 | 15 | 7.41 |

Table 16 shows the evolution of drug substance BET specific surface area for batches 1 and 2 throughout the isolation process. The results confirm that the drug substance BET specific surface area is effectively controlled with the aging step and for all practical purposes remains unchanged after the aging step. The small variations during the rest of the isolation process do not affect the BET specific surface area by more than 10% of its value immediately following after the aging step. The results also show that the removal of solvent by the distillation step has a similar effect as cooling in Example 11, essentially stopping the agglomeration phenomena initiated during the aging step and “locks” the drug substance BET specific surface area.

TABLE 16
Distillation profile.

| | BET SSA | |
|---|---|---|
| | (m²/g) | |
| Sample | Batch 1 | Batch 2 |
| After precipitation | 56.70 | 46.94 |
| After 6 hours at aging temperature | 9.60 | 7.36 |
| After 1% of batch distilled | 9.18 | 7.14 |
| After 2% of batch distilled | 9.13 | 6.90 |
| After 4% of batch distilled | 9.05 | 6.88 |
| After distillation | 8.88 | 6.92 |
| Final dry cake | 9.30 | 7.61 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A drug substance comprising a compound of structural formula I: 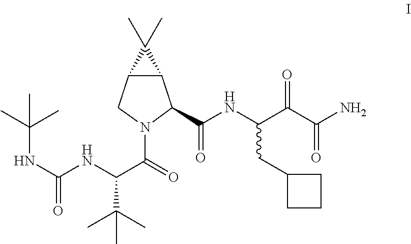 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]
wherein said drug substance is solid and said drug substance has a BET specific surface area of from about 2.9 m²/g to about 94 m²/g.

2. The drug substance according to claim 1, wherein said drug substance has a BET specific surface area of from about 2.9 m²/g to about 9.6 m²/g.

3. The drug substance according to claim 1, wherein said drug substance has a BET specific surface area of from about 2.9 m²/g to about 9.4 m²/g.

4. A pharmaceutical composition comprising at least one drug substance according to claim 1 and at least one pharmaceutically acceptable carrier.

5. The pharmaceutical composition according to claim 4, further comprising at least one excipient.

6. A process for isolating a drug substance, said process comprising:
a) precipitating a compound of structural formula I: 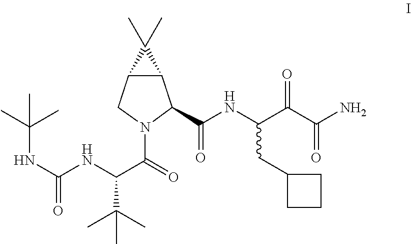 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]
at a temperature below about 5.0° C. from a supersaturated solution to form a slurry;
b) optionally distilling said slurry to form a concentrate;
c) filtering said slurry or concentrate to form a wet cake; and
d) drying said wet cake to form a powder;
wherein:
the powder comprises isolated drug substance; and
said isolated drug substance has a BET specific surface area of from about 2.9 m²/g to about 94 m²/g.

7. The process according to claim 6, wherein said distilling step b) is conducted.

8. The process according to claim 7, wherein said distilling step b) is conducted at a temperature in a range from about −15.0° C. to about 35.0° C.

9. The process according to claim 7, wherein said distilling step b) is conducted at a temperature in a range from about 15.0° C. to about 30.1° C.

10. The process according to claim 7, wherein said distilling step b) is conducted at a temperature in a range from about 15.0° C. to about 24.6° C.

11. The process according to claim 10, wherein said distilling step b) is conducted at a temperature in a range from about 15.1° C. to about 24.6° C. for the first 10 hours of the distilling.

12. The process according to claim 7, wherein said distilling step b) is conducted at a temperature in a range from about −15.0° C. to about 15.0° C.

13. The process according to claim 7, wherein said distilling step b) is conducted over a total of 20 to 30 hours.

14. The process according to claim 6, wherein said filtering step c) is conducted at a temperature in a range from about −15.0° C. to about 15.0° C.

15. A process for isolating a drug substance comprising
a) precipitating compounds of structural formula I: 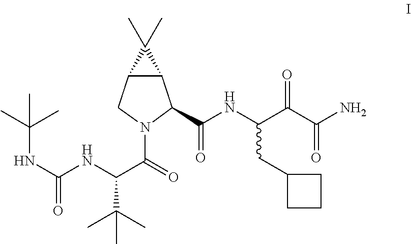 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]
at a temperature below about 5.0° C. from a supersaturated solution to form a slurry;
b) heating said slurry to an aging temperature of between 5.0° C. and 25° C. and holding said slurry at said aging temperature for a period of time;
c) distilling said slurry to form a concentrate, at a temperature of between about −5.0° C. to about 35.0° C., where the distilling temperature is equal or lower to said aging temperature, for the first 4 to 6 hours of distillation;
d) filtering said concentrate to form a wet cake; and
e) drying said wet cake to form a powder; wherein said powder comprises the isolated drug substance; and said isolated drug substance has a BET specific surface area of from about 2.9 m²/g to about 94 m²/g.

16. The process according to claim 15, wherein said heating step b) is conducted at a temperature of between 14° C. and 18° C.

17. The process according to claim 15, wherein said heating step b) comprises holding said slurry at said temperature for 6 hours.

18. The process according to claim 15, wherein said distilling step c) is conducted at a temperature in a range from about 13.0° C. to about 30.1° C.

19. A drug substance prepared by the process according to claim 6.

20. The drug substance according to claim 19, wherein the drug substance has a BET specific surface area of from about 2.9 m²/g to about 9.6 m²/g.

21. A pharmaceutical composition comprising the drug substance according to claim 19 and a pharmaceutically acceptable carrier.

22. The pharmaceutical composition according to claim 21, further comprising at least one excipient.

23-26. (canceled)
